(19) United States
(12) Patent Application Publication
(10) Pub. No.: US 2025/0003995 A1
(43) Pub. Date: Jan. 2, 2025

(54) WET WIPE FOR RAPID DETECTION OF OPIOIDS WITH ALCOHOL AS PRESERVATIVE

(71) Applicants: Abraham Joy, East Walpole, MA (US); Yu Cui, Boston, MA (US)

(72) Inventors: Abraham Joy, East Walpole, MA (US); Yu Cui, Boston, MA (US)

(21) Appl. No.: 18/882,165

(22) Filed: Sep. 11, 2024

Related U.S. Application Data
(63) Continuation-in-part of application No. 17/258,789, filed on Jan. 8, 2021.
(60) Provisional application No. 62/696,484, filed on Jul. 11, 2018.
(60) Provisional application No. 63/684,136, filed on Aug. 16, 2024.

(51) Int. Cl. G01N 33/94; A61K 8/02

(52) U.S. Cl. CPC G01N 33/9486; A61K 8/0208

(57) ABSTRACT

A substance detector includes curcumin and is safe for human handling. The curcumin, as part of a wetting agent of the substance detector, changes color on contact with opioids and related illicit substances. A corresponding method detects very small quantities of an opioid via a rapid color change on a wet wipe when contacted with the opioid. Because the color change takes place on the wet wipe relatively quickly, there is no need to mix reagents to produce a color change reaction.

### CROSS REFERENCE TO RELATED APPLICATIONS

This application is a continuation-in-part of application Ser. No. 17/258,789, filed Jan. 8, 2021, which is a National Stage Entry of International Patent Application No. PCT/US2019/040919, filed Jul. 9, 2019, which claims the benefit of U.S. Provisional Application No. 62/696,484, filed Jul. 11, 2018, all of which applications are incorporated by reference herein. This application also claims the benefit of U.S. Provisional Application No. 63/684,136, filed Aug. 16, 2024, which is incorporated by reference herein.

### TECHNICAL FIELD

This disclosure is directed to a substance detector, which contains a wetting agent material that changes color to indicate the presence of a substance, such as an opioid.

### BACKGROUND

According to the Center for Disease Control (CDC), over one hundred people died every day of 2016 from opioid overdose, accounting for more than 42,000 opioid related deaths in the U.S. that year alone. The annual increase in opioid addiction has been highest in Ohio and New Hampshire, with opioid overdoses increasing 70% from July 2016 to September 2017. First responders to the scenes of these overdoses must be extremely cautious of inadvertent exposure to these potent opioid substances.

Many opioids, and mixtures thereof, are extremely toxic to humans and animals and can cause severe illness and fatalities from small quantities e.g., a few milligrams or even less than 5 micrograms (μg) of certain opioids. In some cases, small quantities of opioids and mixtures thereof, are present on surfaces are not readily visible and can lead to extreme illness, unconsciousness and even death if the opioid comes in contact with compromised skin, inhaled or ingested by adjacent humans and animals. Further, traditional opioid detecting reagents themselves are often dangerous or poisonous to humans.

For instance, carfentanyl has a lethal dose of about 2 milligrams (mg), and thus, particles that inadvertently come in contact with persons in law enforcement, emergency medical services, firefighters, and other first responders could be fatal. In spite of the significant risk, there is currently no quick, cost-effective and rapid technology that enables a first responder to make a confident determination of the hazardous nature of a suspicious drug. Existing technologies for detecting the presence of opioids include Raman/IR detectors and ‘spot test’ kits. Raman and IR detectors are very accurate and can identify samples from a safe distance and without opening the packaging. However, the high cost of these hand-held detectors (˜$20,000) prevents their deployment to every first responder.

Drug-detecting tests, sometimes referred to as spot tests, have been designed to differentiate among various opioids, amphetamines and other illicit drugs. These tests have been developed, modified and optimized to identify closely related classes of illicit drugs and are very useful in the prosecution of a crime. The Marquis reagent, for example, may be employed for the detection of morphine and other alkaloids, which show a red-violet color. This test has also been used as a general screening test for many drugs including various types of amphetamines. Several other tests such as Simon's reagent (for secondary amines), Mecke (for opium alkaloids), Liebermann's (for phenols), Fast blue (for cannabinoids), and Zwikker (for barbiturates) have been developed. These reagents are designed to react with functional groups of the drugs to form color compounds.

Currently, the most common drug-detecting tests come in the form of kits that include sachets that contain various ampules. The user must pick up and transfer the suspected drug to the sachet and break the various ampules to initiate a reaction. The solution changes color and the color itself can be used to identify the drug. These methods can be highly accurate and reproducible. However, these methods require the user to pick up the drug, and this puts the user in danger of exposure that may be potentially fatal in case of highly lethal drugs like carfentanyl. This causes undue safety issues to the first responder who has to carry out the test on-site under high stress conditions. Other drug-detection products use a similar technology along with an applicator or sample collection surface.

Current wipe-based products use a cobalt thiocyanate dye to indicate, by color change, contact with cocaine. Unfortunately, cobalt thiocyanate is toxic, and these wipe products cannot be used on surfaces that may come into human contact.

Other products that are available include test pens. The technology of these test pens is based on antigen-enzyme binding. The small applicator tip of the pen also restricts detection of drugs on large surfaces. Also, aerosolized sprays are available that are also based upon antigen-enzyme binding technology. However, products that rely upon antigen-enzyme binding technology are relatively expensive and may not be accessible to all first responders.

### SUMMARY

This disclosure provides a substance detector including curcumin, and a corresponding method to detect very small quantities of an opioid via a rapid color change on a wet wipe when contacted with an opioid. The substance detector/wet wipe is safe for human handling (i.e., non-toxic). The curcumin, as part of a wetting agent of the wet wipe, changes color on contact with opioids and related illicit substances. Because the color change takes place on the wet wipe almost instantaneously, there is no need to mix reagents to produce a color change reaction.

A product of this disclosure may be termed “wet wipe,” “drug-detecting wipe,” “substance detector,” “substance-detection substrate,” “wetting agent-impregnated wipe,” “drug-detecting glove,” “indicator cloth,” or words of similar import. In one embodiment, the disclosed drug-detecting wipe is a woven or nonwoven substrate that is soaked or impregnated with a color-changing detector, which is a solution comprising curcumin and alcohol, all of which is described in further detail below. In some situations it may be stated that the solution changes color, while in others it may be stated that the drug-detecting wipe changes color; the meaning is the same. The fabric or cloth (nonwoven or woven) of the disclosure, that is, the dry article prior to impregnation with wetting agent, can be called a “substrate.” The term “wetting agent” means the mixture or solution of at least curcumin in alcohol which wets and impregnates the substrate. Other terms that may be used interchangeably with “wetting agent” include “solution,” “detecting solution,” “suspension,” or “color-changing solution.” Several other terms may be used interchangeably, such as “drug,” “illicit substance,” “potentially dangerous substance,” “amine-containing substance,” “opioid,” or any of a number of specific drug names like “cocaine” disclosed elsewhere herein.

In a first aspect, the disclosure provides a fabric, wherein the fabric excludes preservative compounds, wherein the fabric is at least partially saturated with a wetting agent, wherein the wetting agent comprises: curcumin, starch and an alcohol and excludes preservative compounds. In this exemplary embodiment or another exemplary embodiment, the preservative compounds which are excluded include at least one of antioxidants; antimicrobials; chelating agents; natural preservatives; artificial preservatives; vitamin E; vitamin C; butylated hydroxy anisole (BHA); butylated hydroxy toluene (BHT); ascorboyl palmitate; hydroquinone; propyl gallate; alpha-tocopherol; nordihydroguaiaretic acid; lecithin; buffer systems; H₃PO₄/NaH₂PO₄; H₂CO₃/NaHCO₃; sodium paraben; ethyl paraben; propyl paraben; phenols; ethylene diamine tetra acetic acid (EDTA); polyphosphates citric acid; neem oil; lemon extract; lemon oil; limonene; orange oil; orange extract; honey; salts; sodium chloride; chlorocresol; o-phenyl phenol; alkyl esters of p-hydroxy benzoic acid; benzoates; benzoic acid; benzoic acid salts, sorbates; sorbic acid; sorbic acid salts, acetic acid; carboxylic acids; boric acid; boric acid salts; propionates; propionic acid; propionic acid salts; nitrites; and sodium nitrite; quaternary ammonium compounds; benzalkonium chlorides; and cetylpyridinium chlorides. In this exemplary embodiment or another exemplary embodiment, the preservative compounds which are excluded comprise antioxidants; antimicrobials; chelating agents; natural preservatives; artificial preservatives; vitamin E; vitamin C; butylated hydroxy anisole (BHA); butylated hydroxy toluene (BHT); ascorboyl palmitate; hydroquinone; propyl gallate; alpha-tocopherol; nordihydroguaiaretic acid; lecithin; buffer systems; H₃PO₄/NaH₂PO₄; H₂CO₃/NaHCO₃; sodium paraben; ethyl paraben; propyl paraben; phenols; ethylene diamine tetra acetic acid (EDTA); polyphosphates citric acid; neem oil; lemon extract; lemon oil; limonene; orange oil; orange extract; honey; salts; sodium chloride; chlorocresol; o-phenyl phenol; alkyl esters of p-hydroxy benzoic acid; benzoates; benzoic acid; benzoic acid salts, sorbates; sorbic acid; sorbic acid salts, acetic acid; carboxylic acids; boric acid; boric acid salts; propionates; propionic acid; propionic acid salts; nitrites; and sodium nitrite; quaternary ammonium compounds; benzalkonium chlorides; and cetylpyridinium chlorides.

In this exemplary embodiment or another exemplary embodiment, the alcohol comprises ethanol. In this exemplary embodiment or another exemplary embodiment, the wetting agent comprises from about 0.01 to about 5% w/v of curcumin. In this exemplary embodiment or another exemplary embodiment, the wetting agent comprises from about 0.1 to about 2% w/v of curcumin. In this exemplary embodiment or another exemplary embodiment, the starch is selected from the group comprising corn starch, potato starch, wheat starch, rice starch, and combinations thereof. In this exemplary embodiment or another exemplary embodiment, the starch comprises corn starch. In this exemplary embodiment or another exemplary embodiment, the wetting agent comprises from about 0.01 to about 5% w/v starch. In this exemplary embodiment or another exemplary embodiment, the wetting agent further comprises water. In this exemplary embodiment or another exemplary embodiment, ratio of ethanol to water in weight percentage is within a range of 10:1 to 1:10. In this exemplary embodiment or another exemplary embodiment, the wetting agent is present at concentration of from about 0.1 to about 5 grams per 1 gram of dry fabric. In this exemplary embodiment or another exemplary embodiment, the fabric may be woven or nonwoven. In this exemplary embodiment or another exemplary embodiment, the wetting agent comprises curcumin from about 0.05% to about 5% w/v, or about 0.05 to about 0.25% w/v, or about 0.01% to about 1% w/v or about 0.1 to about 0.15% w/v. In this exemplary embodiment or another exemplary embodiment, the wetting agent comprises starch from about 0.01% to about 5% w/v, or about 0.01% to about 1% w/v, or about 0.2 to about 0.5% w/v, or about 0.4 to about 0.8% w/v, or about 0.5 to about 0.7% w/v. In this exemplary embodiment or another exemplary embodiment, the wetting agent is present at concentration of from about 0.1 gram to about 5 grams per 1 gram of dry fabric; or from about 1 gram to about 5 grams per 1 gram of dry fabric; or from about 1 gram to about 3 grams per 1 gram of dry fabric; or from about 1 gram to about 2 grams per 1 gram of dry fabric. In this exemplary embodiment or another exemplary embodiment, the weight ratio of ethanol to water is from about 10:1 to about 1:10; preferably, from about 5:1 to about 1:5 or preferably from about 10:1 to about 1:1.

In another aspect, the disclosure provides a method of detecting opioids or illicit substances comprising: contacting a surface to be tested for illicit substances with any fabric disclosed elsewhere herein, wherein the fabric excludes preservative compounds, wherein the fabric is at least partially saturated with a wetting agent, wherein the wetting agent comprises: curcumin, starch and an alcohol, and excludes preservative compounds; and, determining whether the fabric has changed color, wherein a change in color is indicative of a presence of at least one illicit substance. In this exemplary embodiment or another exemplary embodiment, the preservative compounds which are excluded include antioxidants; antimicrobials; chelating agents; natural preservatives; artificial preservatives; vitamin E, vitamin C; butylated hydroxy anisole (BHA); butylated hydroxy toluene (BHT); ascorboyl palmitate; hydroquinone; propyl gallate; alpha-tocopherol; nordihydroguaiaretic acid; lecithin; buffer systems; H₃PO₄/NaH₂PO₄; H₂CO₃/NaHCO₃; acetic acid; carboxylic acids; sodium paraben; ethyl paraben; propyl paraben; phenols; ethylene diamine tetra acetic acid (EDTA); polyphosphates citric acid; neem oil; lemon extract; lemon oil; limonene; orange oil; orange extract; honey; salts; sodium chloride; chlorocresol; o-phenyl phenol; alkyl esters of p-hydroxy benzoic acid; sorbates; sorbic acid; sorbic acid salts, boric acid; boric acid salts; propionates; propionic acid; propionic acid salts; nitrites; sodium nitrite; quaternary ammonium compounds; benzalkonium chlorides; and cetylpyridinium chlorides.

In another aspect, the disclosure provides a method of detecting opioids or illicit substances comprising: contacting a surface to be tested for illicit substances with any fabric disclosed elsewhere herein; and, determining whether the fabric has changed color, wherein a change in color is indicative of a presence of at least one illicit substance. In this exemplary embodiment or another exemplary embodiment, in the fabric is provided in an airtight package and the method further comprises removing the fabric from the airtight package. In this exemplary embodiment or another exemplary embodiment, method further comprises: visually inspecting the fabric to determine if any portion of the fabric has changed color. In this exemplary embodiment or another exemplary embodiment, method further comprises: In this exemplary embodiment or another exemplary embodiment, the wetting agent comprises a Cₓ alcohol and a C_{y} alcohol where x<5; y<5 and x≠y.

In this exemplary embodiment or another exemplary embodiment, a target analyte is an illicit substance. In this exemplary embodiment or another exemplary embodiment, the target analyte is an opioid. In this exemplary embodiment or another exemplary embodiment, the target analyte is at least one selected from the group comprising: fentanyl, carfentanyl, cocaine, methamphetamine, morphine, hydrocodone, oxycodone, naloxone, endorphins, heroin, codeine, and mixtures and derivatives thereof.

In yet another aspect, the disclosure provides a kit comprising a fabric at least partially saturated with a wetting agent comprising: curcumin, starch and an alcohol. In this exemplary embodiment or another exemplary embodiment, the kit comprises at least 5, at least 6, at least 8, at least 10, at least 12, at least 24, at least 25 or at least 50 of the wipes, or multiples of the foregoing.

In still another aspect, an exemplary embodiment of the present disclosure may provide a nonwoven fabric at least partially saturated with a composition comprising: curcumin, starch and a polar solvent. In this exemplary embodiment or another exemplary embodiment, the polar solvent comprises an alcohol. In this exemplary embodiment or another exemplary embodiment, the alcohol comprises 1 to 6 carbon atoms. In this exemplary embodiment or another exemplary embodiment, the alcohol comprises ethanol. In this exemplary embodiment or another exemplary embodiment, the polar solvent further comprises water. In this exemplary embodiment or another exemplary embodiment, the ratio of ethanol to water in weight percentage is within a range of 10:1 to 1:10. In this exemplary embodiment or another exemplary embodiment, the nonwoven fabric comprises 0.01% to 5% by weight of curcumin. In this exemplary embodiment or another exemplary embodiment, the nonwoven fabric comprises 0.05% to 1% by weight of curcumin. In this exemplary embodiment or another exemplary embodiment, the nonwoven fabric further comprises 0.05% to 5% by weight, preferably 0.05% to 1% by weight, and more preferably 0.1% to 0.15% by weight of curcumin. In this exemplary embodiment or another exemplary embodiment, the nonwoven fabric further comprises 0.1% by weight to 5% by weight, 0.2% by weight to 0.5% by weight of starch, or 0.5% by weight to 0.7% by weight of starch. In this exemplary embodiment or another exemplary embodiment, the starch is selected from corn starch, potato starch, wheat starch, rice starch, and combinations thereof. In this exemplary embodiment or another exemplary embodiment, the starch is modified in a manner selected from the group comprising acid-modified, caustic-modified, oxidized, and emulsified. In this exemplary embodiment or another exemplary embodiment, the starch comprises 15-40 wt. % amylose and 60-85 wt. % amylopectin. In this exemplary embodiment or another exemplary embodiment, the nonwoven fabric further comprises at least one alcohol selected from methanol, ethanol, propanol, butanol, pentanol, and hexanol (where, when such exists, the alcohol may be in its primary, secondary or tertiary forms). In this exemplary embodiment or another exemplary embodiment, the nonwoven fabric may comprise viscose.

In this exemplary embodiment or another exemplary embodiment, the nonwoven fabric further comprises another fiber selected from acetate fibers, acrylic fibers, cellulose ester fibers, cellulose acetate fibers, modacrylic fibers, polyamide fibers, polyester fibers, polyolefin fibers, polyvinyl alcohol fibers, rayon fibers, acrylonitrile-based fibers, polyamide fibers, nylon fibers; polyethylene terephthalate fibers; polyolefin fibers such as polypropylene, polyethylene; polyvinyl acetate fibers; polyurethane fibers and mixtures thereof. In this exemplary embodiment or another exemplary embodiment, the nonwoven fabric has a dry basis density of from about 25 g/m² to about 420 g/m², or 30 g/m² to about 300 g/m², or 50 g/m² to about 125 g/m². In this exemplary embodiment or another exemplary embodiment, the nonwoven fabric has a tribology corresponding to a coefficient of static friction within the range of about 0.1 to 0.9, 0.2 to 0.8 or 0.4 to about 0.6.

In another aspect, the disclosure provides a method of detecting illicit substances comprising: (a) providing a substrate at least partially impregnated with a wetting agent comprising alcohol, curcumin and starch; (b) contacting a surface to be tested for illicit substances with the substrate; and, (c) determining whether the substrate or wetting agent has changed color, wherein a change in color is indicative of a presence of at least one illicit substance.

In another aspect, the disclosure provides a method of detecting opioids comprising: (a) providing a substrate at least partially impregnated with a wetting agent comprising alcohol, curcumin and starch; (b) contacting a surface to be tested for opioids with the substrate; and, (c) determining whether the substrate or wetting agent has changed color, wherein a change in color is indicative of a presence of the opioid. In this exemplary embodiment or another exemplary embodiment, the substrate is provided in an airtight package. In this exemplary embodiment or another exemplary embodiment, the method further comprises removing the substrate from the airtight package. In this exemplary embodiment or another exemplary embodiment, the method further comprises: visually inspecting the substrate to determine if any portion of the substrate has changed color. In this exemplary embodiment or another exemplary embodiment, at least a portion of the substrate or wetting agent undergoes a visible color change within about thirty seconds, or within about ten seconds or within about five seconds, of contact with a target analyte.

In this exemplary embodiment or another exemplary embodiment, the wetting agent/solution comprises a Cₓ alcohol and a C_{y} alcohol where x<5; y<5 and x≠y. In this exemplary embodiment or another exemplary embodiment, the solution further comprises water, wherein the alcohol and water are present in a weight or volume ratio in the range of 10:1 to 1:10. In this exemplary embodiment or another exemplary embodiment, the solution comprises 0.01% to 5% by weight of curcumin, or 0.05% to 1% by weight of curcumin, or about 0.05 to about 0.25% w/v, or 0.1% to 0.15% by weight of curcumin. In this exemplary embodiment or another exemplary embodiment, the solution further comprises 0.1% to 5% starch by weight, or 0.2% to 0.5% starch by weight, or 0.5% by weight to 0.7% by weight of starch. In this exemplary embodiment or another exemplary embodiment, the starch comprises at least one selected from corn starch, potato starch, wheat starch, and rice starch. In this exemplary embodiment or another exemplary embodiment, the starch is at least one of acid-modified, caustic-modified, oxidized, and emulsified. In this exemplary embodiment or another exemplary embodiment, the starch comprises 15-40 wt. % amylose and 60-85 wt. % amylopectin. In this exemplary embodiment or another exemplary embodiment, the solution comprises ethanol and at least one of propanol, butanol, pentanol, and hexanol. In this exemplary embodiment or another exemplary embodiment, the nonwoven fabric comprises viscose. In this exemplary embodiment or another exemplary embodiment, the nonwoven fabric further comprises another fiber selected from the group consisting of acetate fibers, acrylic fibers, acrylonitrile-based fibers, cellulose acetate fibers, cellulose ester fibers, modacrylic fibers, polyamide fibers, nylon fibers, polyester fibers, polyethylene terephthalate fibers, polyolefin fibers polypropylene fibers, polyethylene fibers, polyvinyl alcohol fibers, rayon fibers, polyurethane fibers and mixtures thereof. In this exemplary embodiment or another exemplary embodiment, the nonwoven fabric comprises viscose having a tribology corresponding to a coefficient of static friction within the range of 0.1 to 0.9, 0.2 to 0.8 or 0.4 to about 0.6. In this exemplary embodiment or another exemplary embodiment, the nonwoven fabric is impregnated with a solution comprising curcumin, starch and alcohol.

In this exemplary embodiment or another exemplary embodiment, the substrate comprises a film, a woven fabric, a nonwoven fabric, a cellulose tissue, a paper towel, a coform material, an airlaid material, a polyester amide polymer having pendant functional groups, and/or a bonded-carded web. In this exemplary embodiment or another exemplary embodiment, the substrate comprises natural fibers, synthetic fibers, or combinations thereof. In this exemplary embodiment or another exemplary embodiment, the substrate comprises fibers selected from the group consisting of wood pulp, cotton, cellulose, bamboo, hemp, polyolefin, polyester, polyamide, polylactic acid, rayon, semi-synthetic fiber made from wood pulp sold under the trademark Lyocell™, and combinations thereof. In this exemplary embodiment or another exemplary embodiment, the substrate is in the form of a wipe, a swab, or a glove. In this exemplary embodiment or another exemplary embodiment, the wetting agent comprises at least one of water and a C₁-C₆ alcohol. In this exemplary embodiment or another exemplary embodiment, a method may further comprise: visually inspecting the at least one substrate after the step of contacting, to determine if any portion of the at least one substrate has changed color. This exemplary embodiment or another exemplary embodiment may further provide that at least a portion of the wipe or wetting agent undergoes a visible color change within about thirty seconds of contact with a target analyte. In this exemplary embodiment or another exemplary embodiment, at least a portion of the wipe or wetting agent undergoes a visible color change within about ten seconds of contact with a target analyte. In this exemplary embodiment or another exemplary embodiment, at least a portion of the wipe or wetting agent undergoes a visible color change within about five seconds of contact with a target analyte.

In still another aspect, the disclosure provides a method of making a curcumin-starch solution comprising: preparing (a) a curcumin alcohol solution comprising from about 0.1 to about 50 grams curcumin per liter of alcohol, preparing (b) a starch suspension comprising from about 0.1 to about 300 grams starch per liter of water, and contacting the curcumin alcohol solution with the starch suspension. The (a) curcumin alcohol solution may have a curcumin concentration (in grams curcumin per liter of alcohol) of 0.01-100, 0.1-50, 1-45, 1.2-40, or 2, 4, 5, 10, 15, 20, 25, 30, 35, 40, 45, 50, 55, 60, 65, 70, 75, 80, 85, 90, 95 or 100, or other values in between. The (b) starch suspension may have a starch concentration (in grams starch per liter of water) of 0.01-100, 0.1-50, 5-250, 6-200, or 10, 20, 30, 40, 50, 60, 70, 80, 90, 100, 110, 120, 130, 140, 150, 160, 170, 180, 190, 200, 210, 220, 230, 240, 250, 260, 270, 280, 290, or 300, or other values in between. The (a) curcumin alcohol solution and (b) starch suspension may be combined at a volume ratio in the range of 10:1 to 1:10, for example, 9:1, 8:1, 7:1, 6:1, 5:1, 4:1, 3:1, 2.5:1, 2:1, 1.67:1, 1:1, 1:2, 1:3, 1:4, 1:5, 1:6, 1:7, 1:8, 1:9, or 10:1, or other values in between. The curcumin and starch may be blended together prior to dissolving both in alcohol and diluting with water to achieve desired concentrations of all constituents.

The alcohol may be one or more of methanol, ethanol, n-propanol, iso-propanol, n-butanol, iso-butanol, tert-butanol, n-pentanol, iso-pentanol, tert-pentanol, n-hexanol, iso-hexanol, and tert-hexanol.

Any wipe or other cloth made of a woven or nonwoven material disclosed elsewhere herein may be dipped in, wet with, and/or saturated with any curcumin-starch suspension or solution disclosed elsewhere herein.

### BRIEF DESCRIPTION OF THE DRAWINGS

Sample embodiments of the present disclosure are set forth in the following description, are shown in the drawings, and are particularly and distinctly pointed out and set forth in the appended claims.

FIG. 1 is a top perspective view of a retail container of individually packaged drug-detecting wipes of the disclosure.

FIG. 2 is a front elevation view of a single individually packaged drug-detecting wipe of the disclosure.

FIG. 3A is an operational view of a user donning a pair of protective gloves for use with the drug-detecting wipes of the disclosure.

FIG. 3B is another operational view of the user removing a drug-detecting wipe from its individual package.

FIG. 3C is another operational view of the user moving the drug-detecting wipe across a surface to be tested for illicit substances.

FIG. 3D is another operational view of the user moving a drug-detecting glove across a surface to be tested for illicit substances.

FIG. 4A is a diagrammatic view of the appearance of the drug-detecting wipe of the disclosure with a negative indication (absence) of an illicit substance, for example, an opioid.

FIG. 4B is a diagrammatic view of the appearance of the drug-detecting wipe of the disclosure with a positive indication (presence) of an illicit substance, for example, an opioid.

Similar numbers refer to similar parts throughout the drawings.

### DETAILED DESCRIPTION

Aspects of the disclosure relate to a wet wipe system for rapid detection of an opioid using a specialized color changing ingredient in the presence of the opioid, such as fentanyl, carfentanyl, cocaine, methamphetamine, morphine, hydrocodone, oxycodone, naloxone, endorphins, heroin, codeine, and mixtures and derivatives thereof and other opioids and combinations thereof. The wet wipe formula contains a unique color changing ingredient, curcumin, which can be together with a starch. The curcumin and starch are both within an alcoholic solvent, which may be referred to as being solubilized, to form an alcoholic solution or wetting agent.

In one embodiment, the wet wipe fabric or cloth may be devoid of preservatives as disclosed elsewhere herein. In one embodiment, the wet wipe fabric or cloth may be substantially devoid of preservatives as disclosed elsewhere herein. In one embodiment, the alcoholic solution of curcumin and starch may be devoid of preservatives as disclosed elsewhere herein. In one embodiment, the alcoholic solution of curcumin and starch may be substantially devoid of preservatives as disclosed elsewhere herein.

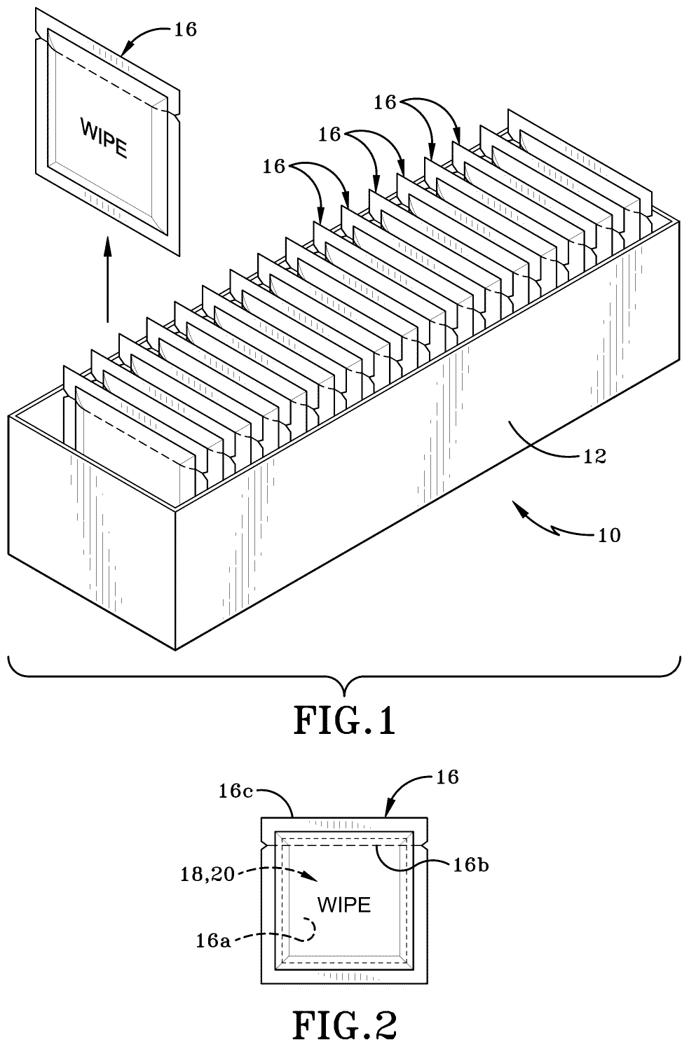

FIG. 1 shows a kit 10 of wipes of the disclosure. Kit 10 includes multi-package (or multi-pack) 12, which may be a retail sized package. Multi-pack 12 holds one or more individual packages (or sachets) 16 of wipe 18. Multi-pack 12 may contain any number of sachets 16 such as 5, 6, 8, 10, 12, 24, 25 or 50, multiples of the foregoing, or other quantities.

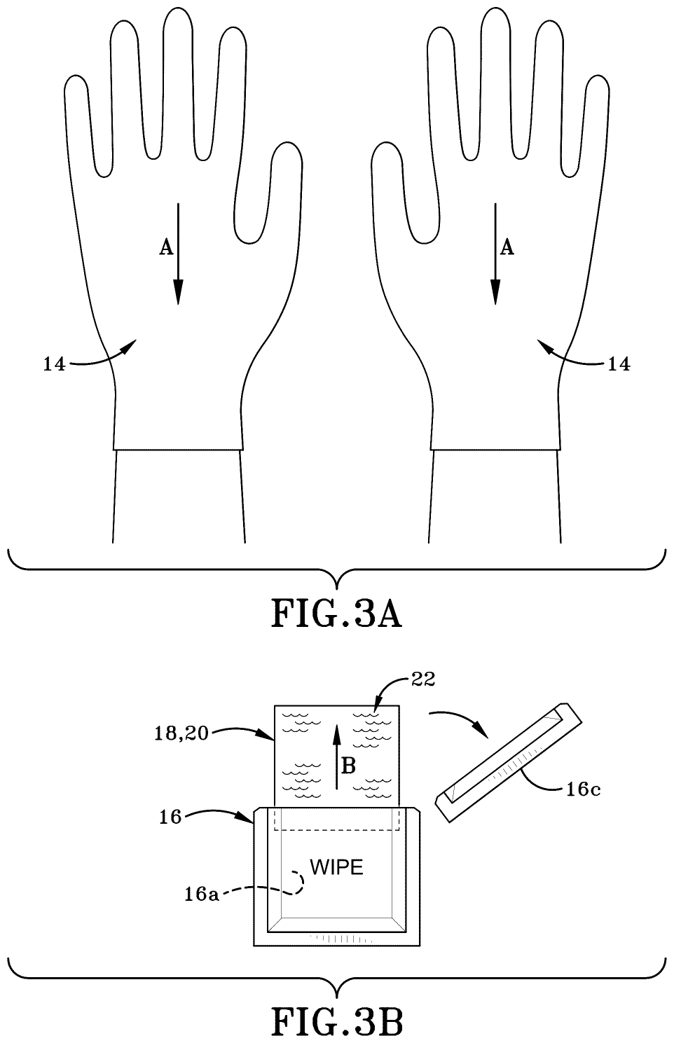

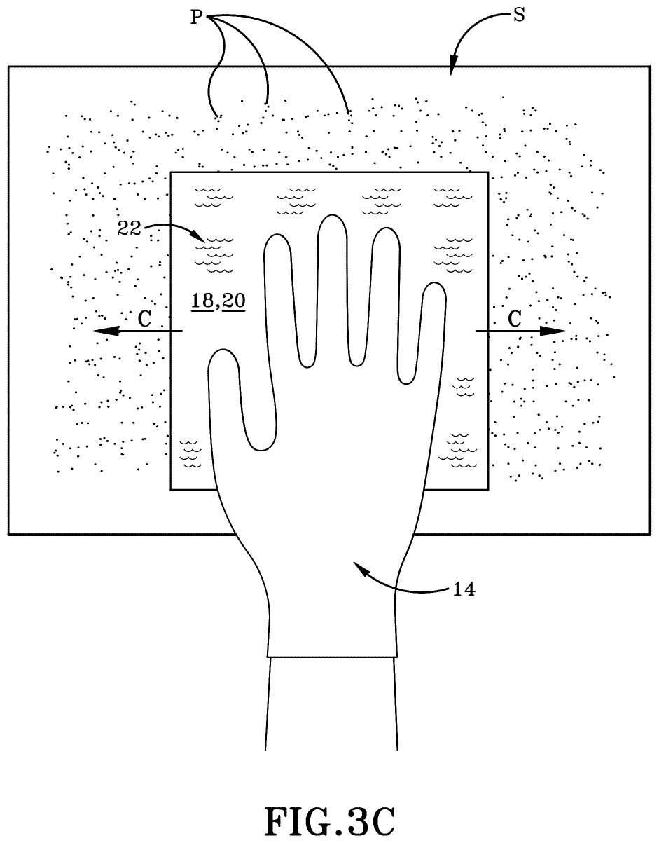

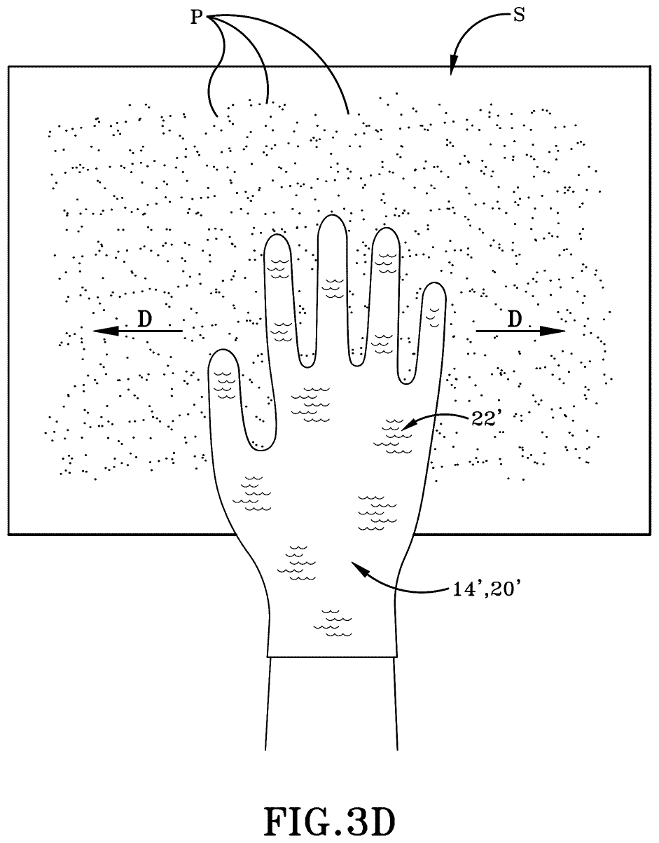

As seen in FIG. 2, in one embodiment, a sachet 16 contains a single wipe 18. Wipe 18 includes substrate 20 and a wetting agent 22 (FIG. 3B) at least partially saturating the substrate. Wipe 18, substrate 20 and wetting agent 22 will be described more fully below.

As seen in FIG. 3A, optionally gloves 14 may be used together with wipe 18. In various embodiments, gloves 14 can be protective gloves, serving no purpose other than protecting a user's hands from potentially harmful substances encountered while using wipe 18 to detect a harmful substance. It is noted that the disclosed drug-detecting wipe 18, substrate 20 and wetting agent 22 are safe for human handling (i.e., non-toxic).

As seen in FIG. 3B and FIG. 2, sachet 16 contains an inner compartment 16a and a line of weakness 16b. Line of weakness 16b allows tear strip 16c to be removed from the remainder of sachet 16 to provide access to wipe 18 within inner compartment 16a. Once tear strip 16c is removed, wipe 18 may be withdrawn from sachet 16 for use.

FIG. 3C operationally shows the drug-detecting wipe 18 being moved back-and-forth over a surface “S” by a user's hand which is covered by glove 14. The drug-detecting wipe 18 may be held in the hand of the user and/or may be pressed against surface “S.” It will be understood that the surface “S” as illustrated in FIG. 3C is representative of any type of surface that may be tested for an illicit substance. For example, the surface “S” may be the hands, face, and/or clothing of a person found at a scene where an illicit substance is being tested for. In other instances, the surface “S” may be a surface of a piece of furniture, a wall or a floor surface, or any other object.

FIG. 3C shows the surface “S” covered with a fine powder “P” of unknown nature. It should be understood that in many instances, the powder “P” may be on the surface “S” but may not be visible to the person gathering evidence from surface “S.” It should further be understood that the powder “P” illustrated in FIG. 3C is an example of any form of potentially illicit substance which may be on surface “S.” For example, instead of solid particles of a potentially illicit substance being on surface “S,” a quantity of a potentially dangerous liquid may be on surface “S.”

The potentially dangerous liquid is to be understood as being represented by powder “P” in FIG. 3C. The wipe 18, as it is moved across surface “S” in the manner indicated by arrows “C,” contacts the powder “P,” and gathers particles of the powder “P” thereon. If the potentially illicit substance is a liquid “P,” then some of that liquid will soak into the wipe 18.

Alternatively, as seen in FIG. 3D, the gloves may be gloves 14′ that may themselves act as the drug-detecting wipe of the disclosure to detect harmful substances. That is, gloves 14′ may include a substrate 20′ (identical to substrate 20) impregnated with wetting agent 22′ (identical to wetting agent 22). Gloves 14′ may have an external layer of absorbent material similar to wipe 18 impregnated with wetting agent 22. Conceptually, gloves 14′ can be thought of as gloves 14 at least partially covered or coated with wipe 18, or its equivalent. In such embodiment, gloves 14′ themselves may be rubbed along surface “S” to detect illicit substances, such as powder “P,” with such motion indicated by arrows labeled “D” in FIG. 3D.

Gloves 14 or gloves 14′ may be fabricated of any suitable glove material which will aid in protecting a person wearing the gloves from being harmed by toxic and/or corrosive substances with which they may come into contact while testing for illicit substances. For example, the material of gloves 14 may aid in protecting the glove-wearer from being harmed by opioids such as fentanyl. One suitable type of glove 14′ which may be useful as the drug-detecting wipe 18 of the disclosure, or as glove 14, which protects a user from illicit substances is any of the gloves described in U.S. Pat. Nos. D910,930; D890,999; 11,825,893; 11,197,509; 11,172,714; 11,071,338; 10,869,512; 10,820,640; 10,820,639; 10,750,802; 10,602,788; 10,602,787; 10,390,575, 10,238,159; 9,968,145; and publication number US 2023/0270192, all of which are assigned to Summit Glove, Inc. of Minerva, Ohio, US and are incorporated herein by reference in their entireties. An additional suitable type of glove 14 or 14′ is any of the gloves described in U.S. patent application Ser. No. 18/488,288 filed Oct. 17, 2023, and entitled “Drug Resistant Glove” which is also assigned to Summit Glove, Inc. of Minerva, Ohio, and is also incorporated herein by reference in its entirety. U.S. patent application Ser. No. 18/488,268, incorporated herein by reference in its entirety, discloses a glove manufactured from a glove material which is opioid resistant. In particular, the glove material is resistant to one or more of fentanyl, carfentanyl, and mixtures of drugs including fentanyl or carfentanyl. Still further, the glove material is resistant to xylazine and to xylazine mixed with an opioid. Yet further, the glove material is resistant to corrosive fluids such as gastric acid.

In operation, and as seen in FIGS. 1, 2, and 3B, the user will remove the sealed sachet 16 containing the wetting agent-impregnated wipe (drug-detecting wipe) 18 from the multi-pack 12. FIG. 2 shows that sachet 16 may include a line of weakness 16b defined in a front and back surface thereof. The user may grip or grasp the tear strip 16c above the line of weakness 16b. Next, the user may tear the tear strip 16c along the line of weakness 16b to access the interior cavity 16a. The user may then grip the wipe 18 from inside the interior cavity 16a. The user may next remove the wipe 18 from the sachet 16, as indicated by arrow “B” in FIG. 3B. Prior to removing the sealed sachet 16, the user may optionally don a pair of gloves 14 as seen in FIG. 3A and in related application Ser. No. 18/591,126, filed 29 Feb. 2024, which is incorporated herein by reference in its entirety. Such donning or wearing of the pair of gloves 14 is denoted by arrows labeled “A” in FIG. 3A.

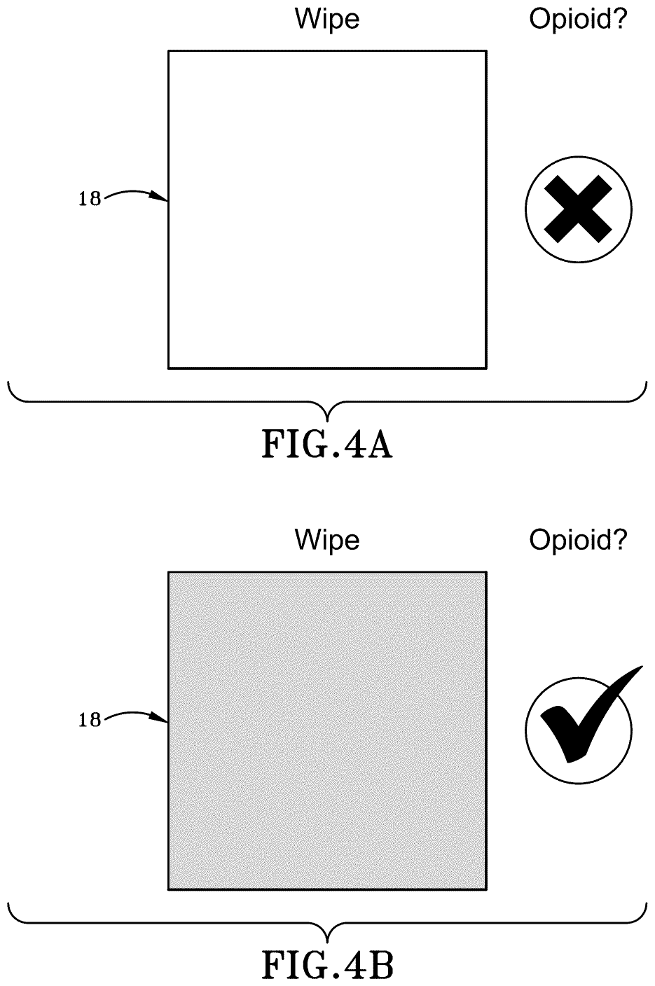

As seen in FIG. 4A, in one example, the color of the substrate 20 will remain unchanged when the treated wipe 18 is wiped across surface “S” and comes into contact with powder “P.” Here, the powder “P” is not a targeted illicit substance, and therefore the color of the substrate 20 remains unchanged, with such state of the substrate 20 being denoted by an “X” that is positioned adjacent to the substrate 20 in FIG. 4A indicating the powder “P” is not a targeted illicit substance.

In another example, the color of the wipe 18 and/or wetting agent 22 will change when the powder “P” is a targeted illicit substance. This is shown in FIG. 4B with the wipe 18 changing color to the darkened color. Such state of the wipe 18 is denoted by a checkmark that is positioned adjacent to the substrate 20 in FIG. 4B indicating the powder “P” is a targeted illicit substance. The color-change signals to the user (e.g., evidence taker) that they will need to take extra caution while gathering further evidence and handling people or objects at the scene.

In FIGS. 3B, 3C, 4A and 4B, a wipe 18 of the disclosure is seen. While a sheetlike square wipe is shown in the Figures, in various embodiments, the disclosed wipe may take any shape. Other possible shapes include rectangular, circular, oval, triangular, or polygons having 4, 5, 6, 7, 8, 9, 10 or more sides. The wipe may also take the shape of a cone, sphere, cube, cuboid, or other polyhedron. The wipe substrate may be adhered to another component, such as a stick, forming a structure similar to a Q-Tip® swab (not shown). In some embodiments, such as shown in FIG. 3D, the substrate 20′ is formed into a glove (or mitten, not shown) allowing the user to wear the substrate 20′ on a hand while detecting the presence of the opioid. As such, the detection can be achieved by movements which the user is accustomed to, such as wiping or rubbing movements.

The wet wipe for rapid detection of opioids can be packaged in many formats such as single sachets, flexible packages, semi-ridged canisters, etc. A plurality of single sachets 16 can be packed into a (container) multi-pack 12 for easy dispensing.

### Curcumin Formulation

Broadly, the curcumin formulation includes curcumin and a solvent. The curcumin formulation may further include starch. The solvent may include an alcohol. The solvent may further include water. The curcumin and starch at least partially dissolve in the alcohol and optionally water. The curcumin and starch dissolved in the alcohol and optionally water may form a solution. Where the curcumin and starch do not fully dissolve, the mixture may be referred to as a suspension. This solution or suspension may be more broadly defined herein as a “wetting agent.”

In one embodiment, the curcumin may be pure curcumin. In another embodiment, the curcumin may contain inevitable impurities including traces of the related compounds demethoxycurcumin and/or bisdemethoxycurcumin, in amounts by weight up to 1%, up to 0.5%, up to 0.3%, or up to 0.1%, or other amounts in between. In further embodiments, other components of turmeric besides the curcuminoids may be present as impurities.

The solution or wetting agent undergoes a color change when it interacts with an illicit substance (e.g., opioids), providing a visual indication of their presence. The color change may occur rapidly, such as within about thirty seconds after contact with an illicit substance, or within about ten seconds after contact with an illicit substance, or within about five seconds after contact with an illicit substance. When opioids are present, they interact with the wetting agent, causing a chemical reaction or complex formation (i.e., between the curcumin and the opioid) that leads to a visible color change. The color change can be easily observed and indicates the presence of opioids on the tested surface.

While not being bound by theory, it is believed that the color change of curcumin upon exposure to a targeted illicit substance is a result of the deprotonation of curcumin when the curcumin is exposed to a pH level of 9 and above. The curcumin, which may also be referred to as an indicator, changes color from yellow to a reddish-brown or wine-red color upon the exposure to an illicit substance, which color change can be seen by the naked eye.

As a concentration level in the solution, the curcumin is present at a level between 0.05% to 5% by weight, or 0.05% to 1% by weight, or 0.05% to 0.25% by weight, or 0.1% to 0.15% by weight of the wetting agent.

Prior to dissolving the curcumin in the alcohol solution, the curcumin may have a particle size distribution of 0.01 to 1 mm; 0.05-0.9 mm; 0.1-0.8 mm; 0.15-0.7 mm; 0.2-0.6 mm; or 0.25-0.5 mm.

### Starch

The curcumin formulation may also include starch. The starch in the curcumin formulation is one or more plant-based starches such as corn starch, potato starch, rice starch, wheat starch, rye starch, chickpea starch, jackfruit starch, preferably corn starch. Amylose constitutes about 15%-30% of common native starches. For example, potato starch contains 11 percent of amylose and corn starch contains 28 percent of amylose. The starch is present at a level of 0.01% to 10% by weight, 0.1% by weight to 5% by weight, or 0.2% by weight to 1% by weight, or 0.2% to 0.5% by weight, or 0.5% to 0.7% by weight of the wetting agent. The starch(es) used in the disclosed formulation may be modified, for example, acid-modified, caustic modified, oxidized, cross-linked, emulsified, etc. The modification of the starch may result in a molecular profile other than that found in nature, such as 15-40 wt. % amylose and 60-85 wt. % amylopectin, or 20-25 wt. % amylose and 75-80 wt. % amylopectin. The amylose may have a degree of polymerization (DP) of about 500 in high-amylose corn starch to more than 6000 DP in potato starch.

The combination of curcumin and starch provides chemical binding to an opioid substance which results in a color-changing chemical reaction between the opioid and curcumin. The color change produced in the curcumin-starch complex by contact with an opioid is unexpected. The curcumin-starch complex dissolved in the wetting agent changes color from a yellowish color to deep red color almost instantaneously.

### Solvent/Solution/Wetting Agent

The carrier or solvent, i.e., wetting agent, includes one or more of short-chain alcohols and water in various combinations. Suitable alcohols include C₁-C₆ alcohols (methanol, ethanol, propanol, butanol, pentanol, hexanol). All forms of the alcohols, n-; iso-; and tert are suitable. Preferably, one or more of methanol, ethanol, and propanol are used as, or as part of, the solvent, with ethanol being most preferable. Within the solvent, an alcohol level greater than 30%; greater than 40%; greater than 50%; greater than 60%; greater than 70%; greater than 80% by weight solubilizes the curcumin in the liquid composition and allows the solubilized curcumin and starch in solution to be evenly distributed on and within the woven or nonwoven material. Further, in one embodiment, the alcohol content of the solution is to be maintained below its flammability limit or flash point.

The alcohol within the solvent, for example, ethanol, may serve an additional purpose beyond solubilizing curcumin. The alcohol may serve as a preservative, for both the curcumin and for the fabric of substrate 20 in wipe 18. When the wipe 18 is withdrawn from sachet 16, the curcumin therein is immediately exposed to the atmosphere, which contains oxygen. Alcohols, for example, ethanol, maintain the curcumin in solution. Without being bound by theory, it is believed that alcohols such as ethanol engage in hydrogen bonding with curcumin, where an H-atom of a solvent ethanol (or water molecule) is H-bonded to an O-atom of curcumin, either at an —OH group or at an —OMe group (either of which is attached to a terminal phenyl group). Alternatively or together with the foregoing, the hydrogen bonding may occur where an H-atom of water or ethanol may H-bond to the oxygen atoms in the central diketone group of curcumin, which is in its enolic configuration, owing to the alcoholic solution.

Alcohols, for example ethanol, can deprive bacteria and viruses of water, thereby depriving such bacteria and viruses of conditions required for life, in one embodiment, reducing water availability. Further, alcohols can cause or accelerate the denaturation (breakdown) of proteins that make up components of bacterial cells or viral capsids (shells), also leading to their death. In this way, alcohols act as bactericides and/or viricides, thus protecting the solubilized curcumin in the wipe, as well as protecting the wipe from any bacteria and/or viruses to which the substrate fabric may be exposed.

There are several major categories of preservative compounds, any or all of which are excluded from the drug-detecting wipe and wetting agents disclosed herein. For example, antioxidants, antimicrobials; chelating agents, natural preservatives and artificial preservatives. There may be some overlap among these categories.

When the overall category of “preservatives” is mentioned herein relative to the exclusion from the drug-detecting wipe and wetting agents disclosed herein, such grouping excludes alcohols, for example, ethanol. Hence, the exclusion of preservatives in certain embodiments herein does not exclude alcohols, such as ethanol, from the wetting agent or fabric of the disclosure.

Exemplary antioxidants that are excluded include vitamin E; vitamin C; butylated hydroxy anisole (BHA); butylated hydroxy toluene (BHT); ascorboyl palmitate; hydroquinone; propyl gallate; alpha-tocopherol; nordihydroguaiaretic acid; lecithin; buffer systems; H₃PO₄/NaH₂PO₄; H₂CO₃/NaHCO₃; and others. At least one of the foregoing is excluded, and preferably, all of the foregoing are excluded.

Exemplary antimicrobial agents that are excluded herein include sodium paraben; ethyl paraben; propyl paraben; phenols; ethylene diamine tetra acetic acid (EDTA); polyphosphates and citric acid. At least one of the foregoing is excluded, and preferably, all of the foregoing are excluded.

Preservatives considered to be “natural” include neem oil; lemon extract; lemon oil; limonene; orange oil; orange extract; honey; salts; sodium chloride; chlorocresol; o-phenyl phenol; alkyl esters of p-hydroxy benzoic acid; benzoates; benzoic acid; benzoic acid salts, sorbates; sorbic acid; sorbic acid salts, acetic acid; carboxylic acids; boric acid; boric acid salts; acetic acid; carboxylic acids; propionates; propionic acid; propionic acid salts; nitrites; and sodium nitrite. At least one of the foregoing is excluded, and preferably, all of the foregoing are excluded.

Further preservatives that are excluded herein include quaternary ammonium compounds; benzalkonium chlorides; and cetylpyridinium chlorides. At least one of the foregoing are excluded, and preferably, all of the foregoing are excluded.

The wetting agent is impregnated into a woven or nonwoven material (“substrate” or “fabric”) which includes natural and/or synthetic fibers as described below. In one embodiment, the amount of wetting agent incorporated into the substrate should maintain the substrate as having a wet nature, while not being dripping wet. In one embodiment, the wetting agent is incorporated into the substrate disclosed herein at a level between 0.1 gram to 10 grams of solution per 1 gram of dry substrate material; or 1 gram to 5 grams of solution per 1 gram of dry substrate material; preferably 1 gram to 3 grams of solution per 1 gram of dry substrate material, more preferably 1 gram to 2 grams of solution per 1 gram of dry substrate material. This embodiment results in very low amounts of wetting agent being transferred to a surface under analysis, where the rapid color change in the presence of an illicit substance, for example, an opioid, takes place on the wipe.

### Fabric/Substrate

A variety of woven and nonwoven material compositions are suitable for performance as the substrate 20 of the disclosed wipe. Certain embodiments employ nonwoven substrates since they are economical and readily available in a variety of materials. However, woven substrates are also suitable. Nonwoven substrates may include a variety of materials both natural and synthetic. An exemplary material includes pulp or cellulosic or natural fibers at a rate of at least 40 wt %, at least 45 wt %, at least 50 wt %, at least 55 wt %, at least 60 wt %, at least 65 wt %, at least 70 wt %, at least 75 wt %, at least 80 wt %, at least 85 wt %, at least 90 wt %, at least 95 wt %, or at least 99 wt %, of the total fiber composition. Without being bound by theory, the curcumin and starch are believed to ionically and lipophilically bind to pulp and cellulosic fibers and combinations thereof to provide a color change on or in the substrate material when the curcumin and starch combination comes in contact with an opioid or other illicit substances or drugs.

The substrate 20 should have sufficient mechanical strength and good processability, and should not be soluble in alcoholic or aqueous medium. In one or more embodiments, the substrate 20 is characterized by a dry basis weight of from about 25 g/m² to about 420 g/m², in other embodiments, from about 30 g/m² to about 300 g/m², in other embodiments, from about 35 g/m² to about 250 g/m² in other embodiments, from about 40 g/m² to about 200 g/m², in other embodiments, from about 45 g/m² to about 150 g/m², and in still further embodiments, from about 50 g/m² to about 125 g/m². The fibers or threads from which the nonwoven substrate is produced may have a denier (linear density) of 1 to 1000 grams, or 10 to 500 grams, or 50 to 250 grams or 100 to 200 grams. It will be appreciated that “denier” is a measure of the weight of 9000 meters of the thread.

In an embodiment, one or more of wood pulp, pulps of other plants, cellulose, and cotton, may be combined with synthetic fibers such as polypropylene, polyethylene, polyesters, polyolefins, etc. or a combination thereof. A preferred nonwoven composition is 100% viscose fibers which delivers acceptable performance.

The term “nonwoven” generally means the material is comprised of fibers which are not woven or knitted together, but rather are held together by another technique such as one or more of bonding, interlocking, chemical, mechanical, heat, or solvent treatments. Nonwoven materials can be formed into a membrane, sheet, substrate, mat, absorbent core or pad layer or combinations thereof.

In one or more embodiments, the substrate 20 may comprise an air-laid nonwoven web. Examples include meltblown, spunbond, and bonded-carded web materials. Air-laid nonwoven sheets, and methods of making the same, are described in U.S. Pat. Nos. 3,849,241, 4,340,563, 4,443,513, 4,548,856, 4,853,281, 5,382,400, 5,575,874, 6,224,977, 6,811,638, 6,946,413, and U.S. Pat. App. Pub. Nos. 2004/0191136 A1 and 2006/0008611 A1, all of which are incorporated herein by reference.

In one or more embodiments, the substrate 20 may comprise a coform nonwoven web. Coform nonwoven webs may be formed by the comingling of polymeric fibers and absorbent fibers, such as polyolefin fibers and cellulosic fibers, as the fibers are entrained by a common airstream before they are deposited onto a forming surface. Coform sheet materials are described U.S. Pat. Nos. 4,100,324 and 5,350,624, and U.S. Pat. App. Pub. Nos. 2011/151596 A1, all of which are incorporated herein by reference. In one or more embodiments, the coform sheet may comprise a matrix of thermoplastic polymeric meltblown fibers and wood pulp fibers, or more generally, natural and manmade fibers.

In one or more embodiments, the substrate 20 may comprise hydroentangled nonwoven sheet materials. Hydroentangled nonwoven webs are sometimes referred to as spunlace fabrics. In certain aspects, hydroentangling readily allows for the combination of different fiber types, such as combining fibers of distinct composition (e.g. polymeric fibers and wood pulp fibers) or fibers of different sizes. Hydroentangled materials, and methods of making the same, are described U.S. Pat. Nos. 3,485,706, 3,620,903, 5,009,747, 5,284,703, and 6,200,669, all of which are incorporated herein by reference.

The term “natural” is meant as materials which are derived from plants, or plant byproducts. The term “synthetic” is meant as fibers and materials which are obtained primarily from various man-made materials or from natural materials which have been further altered. A conventional base starting material is usually a fibrous web comprising any of the common synthetic or natural fibers, or mixtures thereof. Non-limiting examples of natural materials useful in the present disclosure are pulp and cellulosic fibers. Non-limiting examples of cellulosic fibers include those selected from the group consisting of wood pulp fibers, bamboo fibers, cotton fibers, hemp fibers, jute fibers, flax fibers, and mixtures thereof. Non-limiting examples of synthetic materials useful in the present disclosure include those selected from the group consisting of acetate fibers, acrylic fibers, cellulose ester fibers, modacrylic fibers, polyamide fibers, polyester fibers, polyolefin fibers, polyvinyl alcohol fibers, rayon fibers, and mixtures thereof. Examples of some of these synthetic materials include acrylics such as Acrilan®, Creslan®, and the acrylonitrile-based fiber, orlon; cellulose ester fibers such as cellulose acetate, Arnel®, and Acele®; polyamides such as nylons; polyesters such as Fortrel®, Kodel®, and the polyethylene terephthalate fiber, Dacron®; VISTAMAXX® clastic olefin copolymer resin (available from ExxonMobil Corporation) or KRATON® G styrene-ethylene/butylene-styrene and styrene-ethylene/propylene-styrene polymer resins (available from Kraton® Performance Polymers, Inc.); polyolefins such as polypropylene, polyethylene; polyvinyl acetate fibers; polyurethane foams and mixtures thereof. All trademarks are the property of their respective owners. No ownership or affiliation is implied by use of trademarks herein.

It has generally been found that substrates made primarily of synthetic fibers, such as polyethylene terephthalate, polypropylene, polyethylene or a combination thereof, do not bind the curcumin and starch combined in the liquid composition and therefore do not retain the wetting agent to provide an instant color change on the substrate material. While such synthetic materials may form a portion of the substrate fibers, they are not preferred. One example of a more suitable substrate material is 100% viscose fibers. Viscose fibers may be considered both natural and synthetic owing to a number of factors. For purposes of this disclosure, viscose is considered to be a natural fiber.

### Substrate Additives

In one or more embodiments, the substrate 20 of the drug-detecting wipes 18 may further include one or more optional ingredients selected from, but not limited to, color enhancers, catalysts, mechanical property modifiers, optical brighteners, anti-static agents, flame retardants, lubricants, wetting agents, softeners, mordants, and inorganic additives. The optional ingredients may be embedded within, absorbed into, dispersed upon or within, or coated upon the substrate. Chemicals and additives that are typically employed in dyeing processes and methods may be employed with the drug-detecting wipe of the present disclosure, including one or more of soda ash fixer, urea, Dharma™ dye fixative, Synthrapol™, Dharma™ professional textile detergent, Milsoft™, sodium alginate, Superclear™, Calsolene™ oil, Bleach-Stop™, Dharma™ color remover, optic whitener, ammonium sulfate, Jacquard™ silk salt, Retayne™, Ludigal™ F, Dharma™ discharge paste, alum, potassium alum, and Fiber etch.

In one or more embodiments, the drug-detecting wipe includes titanium dioxide. The titanium dioxide may advantageously function as an optical brightener in the drug-detecting wipe disclosed herein. Other inorganic additives may include zeolites, alumina and silica. In one or more embodiments, inorganic additives may be selected to increase the selectivity of the drug-detecting wipe. In one or more embodiments, the substrate may be a composite of an inorganic-polymer matrix embedded with one or more chemicals to provide indication of the presence of opioids. In one or more embodiments, the substrate may be fabricated from polymers and silica particles.

In light of the foregoing, it should be appreciated that the present disclosure advances the art by providing improvements for rapid detection of opioids. While particular embodiments have been disclosed in detail herein, it should be appreciated that the disclosure is not limited thereto or thereby inasmuch as variations on the disclosure herein will be readily appreciated by those of ordinary skill in the art. The scope shall be appreciated from the claims that follow.

### EXAMPLES

### Example 1

A first example for preparing an indicator cloth/wipe utilized ingredients of curcumin (CAS #: 458-37-7), starch (CAS #: 9005-25-8), ethanol, and deionized water.

In a first method (Method 1), 40 g/L curcumin in ethanol was prepared and 200 g/L corn starch in water was prepared. In both mixtures, the solute was not fully dissolved, so they can more accurately be referred to as suspensions. The suspensions were mixed together at a volume ratio of 2.5:1 (curcumin: corn starch).

In a second method (Method 2), curcumin and corn starch powders were mixed directly together and then 70% ethanol was added. In the specific example prepared, 400 mg curcumin and 800 mg corn starch were mixed in a petri dish (in order to provide easier eventual dipping of the mat substrate). 14 mL of 70% ethanol was added to the petri dish and the dish was swirled to dissolve the bulk of the powder.

The mixtures of both Method 1 and Method 2 result in a yellow or yellow-orange solution. After dipping a woven or nonwoven cloth in the mixtures/solutions, the cloth should take on a yellow or yellow-orange color. Then the dipped cloth can be contacted with an opioid for detection thereof. When contacted with the mixtures produced by Method 1 and Method 2 above, an opioid produces a red color on the cloth.

### Example 2

A second example was prepared with ingredients of curcumin (CAS #: 458-37-7), starch (CAS #: 9005-25-8), ethanol, and deionized water.

In a first method (Method A), two separate solutions were prepared with the following ratios: 1.2 g/L curcumin in ethanol (0.12 wt. %) and 6 g/L corn starch in deionized water (0.6 wt. %). For the starch solution, the solute may not be fully dissolved. This can be improved by shaking well to form a suspension, in order to more evenly disperse the starch prior to mixing with the curcumin solution. Then the two solutions were mixed together at a volume ratio of 5:3 (curcumin: corn starch). The overall mixture can be used to prepare an indicator cloth/wipe.

In a second method (Method B), the curcumin solution can first be prepared followed by stirring the starch into the curcumin solution directly. The corresponding amount of water can then be added concurrently or subsequently. The combined mixture can then be mixed well before being used to prepare an indicator cloth/wipe. The Method B includes using the amounts and concentrations set forth in Method A above.

Various modifications and alterations that do not depart from the scope and spirit of this invention will become apparent to those skilled in the art. This invention is not to be duly limited to the illustrative embodiments set forth herein.

What is claimed is:

1. A fabric at least partially saturated with a wetting agent comprising curcumin, starch, and an alcohol, wherein the fabric excludes preservative compounds, and wherein the wetting agent excludes preservative compounds.

2. The fabric of claim 1, wherein the preservative compounds which are excluded include at least one of antioxidants; antimicrobials; chelating agents; natural preservatives; artificial preservatives; vitamin E; vitamin C; butylated hydroxy anisole (BHA); butylated hydroxy toluene (BHT); ascorboyl palmitate; hydroquinone; propyl gallate; alpha-tocopherol; nordihydroguaiaretic acid; lecithin; buffer systems; H₃PO₄/NaH₂PO₄; H₂CO₃/NaHCO₃; sodium paraben; ethyl paraben; propyl paraben; phenols; ethylene diamine tetra acetic acid (EDTA); polyphosphates citric acid; neem oil; lemon extract; lemon oil; limonene; orange oil; orange extract; honey; salts; sodium chloride; chlorocresol; o-phenyl phenol; alkyl esters of p-hydroxy benzoic acid; benzoates; benzoic acid; benzoic acid salts, sorbates; sorbic acid; sorbic acid salts, boric acid; boric acid salts; propionates; propionic acid; propionic acid salts; nitrites; and sodium nitrite; quaternary ammonium compounds; benzalkonium chlorides; and cetylpyridinium chlorides.

3. The fabric of claim 2, wherein the preservative compounds which are excluded comprise antioxidants; antimicrobials; chelating agents; natural preservatives; artificial preservatives; vitamin E; vitamin C; butylated hydroxy anisole (BHA); butylated hydroxy toluene (BHT); ascorboyl palmitate; hydroquinone; propyl gallate; alpha-tocopherol; nordihydroguaiaretic acid; lecithin; buffer systems; H₃PO₄/NaH₂PO₄; H₂CO₃/NaHCO₃; sodium paraben; ethyl paraben; propyl paraben; phenols; ethylene diamine tetra acetic acid (EDTA); polyphosphates citric acid; neem oil; lemon extract; lemon oil; limonene; orange oil; orange extract; honey; salts; sodium chloride; chlorocresol; o-phenyl phenol; alkyl esters of p-hydroxy benzoic acid; benzoates; benzoic acid; benzoic acid salts, sorbates; sorbic acid; sorbic acid salts, boric acid; boric acid salts; propionates; propionic acid; propionic acid salts; nitrites; and sodium nitrite; quaternary ammonium compounds; benzalkonium chlorides; and cetylpyridinium chlorides.

4. The fabric of claim 1, wherein the alcohol comprises ethanol.

5. The fabric of claim 1, wherein the wetting agent comprises from about 0.01 to about 5% w/v of the curcumin.

6. The fabric of claim 1, wherein the fabric is woven or nonwoven.

7. The fabric of claim 1, wherein the wetting agent comprises from about 0.01 to about 5% w/v of the starch.

8. The fabric of claim 1, wherein the wetting agent further comprises water.

9. The fabric of claim 8, wherein the alcohol comprises ethanol, wherein the ratio of ethanol to water in weight percentage is within a range of 10:1 to 1:10.

10. The fabric of claim 1, wherein the wetting agent is present at concentration of from about 0.1 to about 5 grams per 1 gram of dry fabric.

11. A method of detecting illicit substances comprising:
contacting a surface to be tested for illicit substances with a fabric, wherein the fabric excludes preservative compounds, wherein the fabric is at least partially saturated with a wetting agent, wherein the wetting agent comprises curcumin, starch, and an alcohol, and excludes preservative compounds; and
determining, after the step of contacting, whether the fabric has changed color, wherein a change in color is indicative of a presence of at least one illicit substance.

12. The method of claim 11, wherein the preservative compounds which are excluded include at least one of antioxidants; antimicrobials; chelating agents; natural preservatives; artificial preservatives; vitamin E; vitamin C; butylated hydroxy anisole (BHA); butylated hydroxy toluene (BHT); ascorboyl palmitate; hydroquinone; propyl gallate; alpha-tocopherol; nordihydroguaiaretic acid; lecithin; buffer systems; H₃PO₄/NaH₂PO₄; H₂CO₃/NaHCO₃; sodium paraben; ethyl paraben; propyl paraben; phenols; ethylene diamine tetra acetic acid (EDTA); polyphosphates citric acid; neem oil; lemon extract; lemon oil; limonene; orange oil; orange extract; honey; salts; sodium chloride; chlorocresol; o-phenyl phenol; alkyl esters of p-hydroxy benzoic acid; benzoates; benzoic acid; benzoic acid salts, sorbates; sorbic acid; sorbic acid salts, boric acid; boric acid salts; propionates; propionic acid; propionic acid salts; nitrites; and sodium nitrite; quaternary ammonium compounds; benzalkonium chlorides; and cetylpyridinium chlorides.

13. The method of claim 12, wherein the preservative compounds which are excluded comprise antioxidants; antimicrobials; chelating agents; natural preservatives; artificial preservatives; vitamin E; vitamin C; butylated hydroxy anisole (BHA); butylated hydroxy toluene (BHT); ascorboyl palmitate; hydroquinone; propyl gallate; alpha-tocopherol; nordihydroguaiaretic acid; lecithin; buffer systems; H₃PO₄/NaH₂PO₄; H₂CO₃/NaHCO₃; sodium paraben; ethyl paraben; propyl paraben; phenols; ethylene diamine tetra acetic acid (EDTA); polyphosphates citric acid; neem oil; lemon extract; lemon oil; limonene; orange oil; orange extract; honey; salts; sodium chloride; chlorocresol; o-phenyl phenol; alkyl esters of p-hydroxy benzoic acid; benzoates; benzoic acid; benzoic acid salts, sorbates; sorbic acid; sorbic acid salts, boric acid; boric acid salts; propionates; propionic acid; propionic acid salts; nitrites; and sodium nitrite; quaternary ammonium compounds; benzalkonium chlorides; and cetylpyridinium chlorides.

14. The method of claim 11, wherein the alcohol comprises ethanol.

15. The method of claim 14, wherein the wetting agent comprises from about 0.01 to about 5% w/v of the curcumin.

16. The method of claim 11, wherein the fabric is woven or nonwoven.

17. The method of claim 11, wherein the wetting agent comprises from about 0.01 to about 5% w/v of the starch.

18. The method of claim 11, wherein the wetting agent further comprises water.

19. The method of claim 18, wherein the alcohol comprises ethanol, wherein the ratio of ethanol to water in weight percentage is within a range of 10:1 to 1:10.

20. The method of claim 11, wherein the wetting agent is present at concentration of from about 0.1 to about 5 grams per 1 gram of the fabric as a dry fabric.
